(19) United States
(12) Patent Application Publication
(10) Pub. No.: US 2010/0221247 A1
(43) Pub. Date: Sep. 2, 2010

(54) AGENTS AND METHODS FOR TREATMENT OF CANCER

(76) Inventors: Robert BENDER, Ottawa (CA); Charles H. GRAHAM, Battersea (CA); Christine D. COPPLE, Potomac, MD (US)

Correspondence Address: LAHIVE & COCKFIELD, LLP;FLOOR 30, SUITE 3000, ONE POST OFFICE SQUARE, BOSTON, MA, 02109, US

(21) Appl. No.: 12/569,289

(22) Filed: Sep. 29, 2009

Related U.S. Application Data
(60) Provisional application No. 61/100,825, filed on Sep. 29, 2008.
(60) Provisional application No. 61/177,845, filed on May 13, 2009.

(51) Int. Cl. A61K 39/395; A61K 38/00; A61K 31/7088; A61K 31/506; A61K 31/454; A61K 31/4439; A61K 31/4412; A61K 31/404; A61P 35/00

(52) U.S. Cl. 424/133.1; 514/2; 514/44 R; 514/275; 514/323; 514/338; 514/350; 514/414

(57) ABSTRACT

The present application describes compositions that are useful for the treatment, prevention and/or amelioration of cancer.

### RELATED APPLICATIONS

This application claims priority to U.S. Provisional Application No. 61/100,825, Attorney Docket No. NMI-007-1, filed Sep. 29, 2008, titled “AGENTS AND METHODS FOR TREATMENT OF CANCER.” This application also claims priority to U.S. Provisional Application No. 61/177,845, Attorney Docket No. NMI-007-2, filed May 13, 2009, titled “AGENTS AND METHODS FOR TREATMENT OF CANCER.” The contents of any patents, patent applications, and references cited throughout this specification are hereby incorporated by reference in their entireties.

### BACKGROUND OF THE INVENTION

One in every four deaths in the United States is due to cancer, and cancer is the second leading cause of death in the U.S. (U.S. Cancer Statistics Working Group; United States Cancer Statistics: 2000 Incidence, Atlanta (Ga.): Department of Health and

Human Services, Centers for Disease Control and Prevention, and National Cancer Institute (2003)). The National Cancer Institute reports that almost 10 million Americans have a history of invasive cancer. More than 1.43 million people in the United States were expected to be diagnosed with cancer in 2008 and 565,000 people were expected to die, according to projections from the National Cancer Institute (http://seer.cancer.gov/statfacts/html/all.html). Despite the increase in five-year survival rates from 51% in 1975-1977 to 66% in 1996-2002 (American Cancer Society. Cancer Facts & Figures 2007), produced with earlier diagnosis and improved treatments, the death rate per 100,000 people has only gone down 5% since 1950 due to the increased incidence of several types of cancer over the same period (SEER Cancer Statistics Review 1975-2004, NCI “55-Year Trends in U.S. Cancer Death Rates”).

Cancers are classified based on the organ and cell tissue from which the cancer originates, including: (i) carcinomas (most common kind of cancer which originates in epithelial tissues, the layers of cells covering the body's surface or lining internal organs and various glands); (ii) leukemias (origination in the blood-forming tissues, including bone marrow, lymph nodes and the spleen); (iii) lymphomas (originates in the cells of the lymph system); (iv) melanomas (originates in the pigment cells located among the epithelial cells of the skin); and (v) sarcomas (originates in the connective tissues of the body, such as bones, muscles and blood vessels). (See Molecular Biology of the Cell: Third Edition, “Cancer,” Chapter 24, pp. 1255-1294, B. Alberts et al., (eds.), Garland Publishing, Inc., New York (1994); and Stedman's Pocket Medical Dictionary; Williams and Wilkins, Baltimore (1987)). Within these broad cancer classifications, there are over one hundred cancer subclassifications, such as breast, lung, pancreatic, colon, and prostate cancer.

Accordingly, there is a need for developing new and alternative treatments for cancers, including management of cancer and cancer-related disorders.

### SUMMARY OF THE INVENTION

There remains a need for new treatments and therapies useful in the treatment, prevention and/or amelioration of one or more symptoms of cancer, including, but not limited to, adjuvant/non-metastatic colon cancer, metastatic breast cancer, metastatic renal cell carcinoma, metastatic glioblastoma multiforme, metastatic ovarian cancer, metastatic hormone-refractory prostate cancer, and metastatic or unresectable locally advanced pancreatic cancer.

In one aspect, the invention provides a pharmaceutical composition comprising an anti-VEGF antibody and a nitric oxide mimetic for the treatment of cancer. In another aspect, the invention provides a pharmaceutical composition comprising an anti-VEGF antibody and nitroglycerin. In still another aspect, the invention provides a pharmaceutical composition comprising Avastin and a nitric oxide mimetic.

In one embodiment of the composition of the invention, the nitric oxide mimetic is selected from the group consisting of nitroglycerin (GTN), isosorbide 5-mononitrate (ISMN), isosorbide dinitrate (ISDN), pentaerythritol tetranitrate (PETN), erthrityl tetranitrate (ETN), N-hydroxyl-L-arginine (NOHA), N⁶-(1-iminoethyl)lysine) (L-NIL), L-N⁶-(1-iminoethyl)ornithine (LN-NIO), N⁶-methyl-L-arginine (L-NMMA), and S-nitrosoglutathione (SNOG). In another embodiment, the nitric oxide mimetic is selected from the group consisting of S,S-dinitrosodithiol (SSDD), [N-[2-(nitroxyethyl)]-3-pyridinecarboxamide (nicorandil), sodium nitroprusside (SNP), S-nitroso-N-acetylpenicilamine (SNAP), 3-morpholino-sydnonimine (SIN-1), molsidomine, DEA-NONOate(2-(N,N-diethylamino)-diazenolate-2-oxide), and spermine NONOate (N-[4-[1-(3-aminopropyl)-2-hydroxy-2-nitrosohydrazino]butyl-1,3-propanediamine). In still another embodiment, the nitric oxide mimetic is nitroglycerin (GTN).

In another embodiment of the composition, the nitric oxide mimetic is administered using a transdermal patch. In still anther embodiment, the nitric oxide mimetic is administered in a low dose. In one embodiment, the low dose is 3 to 10,000 fold lower than a dose of said nitric oxide mimetic that produces vasodilation. In one embodiment of the low dose, the low dose does not induce substantial tolerance in the subject. In still another embodiment, the low dose is delivered by said nitric oxide mimetic at a concentration between about 10⁻¹⁴ M to about 10⁻⁶ M.

In another aspect, the invention provides a method of treating cancer in a subject in need thereof by administering to the subject a pharmaceutical composition comprising Avastin and a nitric oxide mimetic. In one embodiment, the cancer is selected from the group consisting of breast, ovarian, prostate, lung, colon, rectal, thyroid, bone, testicular, endometrial, bladder, pancreatic and gastrointestinal cancers. In another embodiment, the cancer is selected from the group consisting of breast, ovarian, prostate, lung, colon, and rectal cancer. In still another embodiment, the cancer is metastatic carcinoma of the colon or rectum. In still another embodiment, the cancer is recurrent or metastatic non-squamous, non-small cell lung cancer.

In one embodiment of treating the cancer in a subject, the pharmaceutical composition is administered with an amount of at least one second compound, said second compound being an anti-cancer agent; wherein the amounts of the first composition and said second compound result in a therapeutic effect. In still another embodiment, the second compound is taxol, 5-fluorouracil, paclitaxel, bolus-IFL, FOLFOX4, irinotecan, leucovorin, carboplatin or paclitaxel.

In another aspect, the invention provides a method of treating cancer in a subject in need thereof by administering to the subject a pharmaceutical composition comprising an anti-VEGF antibody and a nitric oxide mimetic. In yet another embodiment, the invention provides a method of treating cancer in a subject in need thereof by administering to the subject a pharmaceutical composition comprising an anti-VEGF antibody and nitroglycerin. In still another embodiment, the invention provides a method of treating cancer in a subject in need thereof by administering to the subject a pharmaceutical composition comprising Avastin and a nitric oxide mimetic, wherein Avastin is administered first followed by administration of a nitric oxide mimetic. In another embodiment of treating the cancer, the nitric oxide mimetic is administered first followed by administration of Avastin. In still another embodiment, Avastin and the nitric oxide mimetic are administered simultaneously. In another embodiment of treating the cancer, the nitric oxide mimetic is selected from the group consisting of nitroglycerin (GTN), isosorbide 5-mononitrate (ISMN), isosorbide dinitrate (ISDN), pentaerythritol tetranitrate (PETN), erthrityl tetranitrate (ETN), N-hydroxyl-L-arginine (NOHA), N⁶-(1-iminoethyl)lysine) (L-NIL), L-N⁶-(1-iminoethyl)ornithine (LN-NIO), N⁶-methyl-L-arginine (L-NMMA), and S-nitrosoglutathione (SNOG). In still another embodiment of treating the cancer, the nitric oxide mimetic is selected from the group consisting of S,S-dinitrosodithiol (SSDD), [N-[2-(nitroxyethyl)]-3-pyridinecarboxamide (nicorandil), sodium nitroprusside (SNP), S-nitroso-N-acetylpenicilamine (SNAP), 3-morpholino-sydnonimine (SIN-1), molsidomine, DEA-NONOate(2-(N,N-diethylamino)-diazenolate-2-oxide), and spermine NONOate (N-[4-[1-(3-aminopropyl)-2-hydroxy-2-nitrosohydrazino]butyl -1,3-propanediamine). In still another embodiment of treating the cancer, the nitric oxide mimetic is nitroglycerin (GTN).

In another embodiment of treating the cancer, the nitric oxide mimetic is administered using a transdermal patch. In still another embodiment of treating the cancer, the nitric oxide mimetic is administered in a low dose. In yet another embodiment of treating the cancer, the low dose is 3 to 10,000 fold lower than a dose of said nitric oxide mimetic that produces vasodilation. In one embodiment of the low dose, the low dose does not induce substantial tolerance in the subject. In still another embodiment of the low dose, the low dose is delivered by said nitric oxide mimetic at a concentration between about 10⁻¹⁴ M to about 10⁻⁶ M. In another embodiment of treating the cancer, the Avastin is administered intravenously.

In another aspect, the invention provides a method of treating cancer in a subject in need thereof by administering to the subject a pharmaceutical composition comprising Avastin and nitroglycerin. In still another aspect, the invention provides a method of treating cancer in a subject in need thereof by administering to the subject a pharmaceutical composition comprising Avastin and a low dose of nitroglycerin. In yet another aspect, the invention provides a method of treating cancer in a subject in need thereof by administering to the subject a pharmaceutical composition comprising Avastin and a low dose of nitroglycerin, wherein the nitroglycerin is administered transdermally. In still another aspect, the invention provides a method of treating cancer in a subject in need thereof by administering to the subject a pharmaceutical composition comprising Avastin and a low dose of nitroglycerin, wherein the nitroglycerin is administered transdermally, and wherein the cancer is breast, prostate, colon or rectal cancer. In still another aspect, the invention provides a method of treating non-small cell lung cancer in a subject in need thereof by administering to the subject a pharmaceutical composition comprising Avastin and a low dose of nitroglycerin, wherein the nitroglycerin is administered transdermally, and wherein the cancer is breast, prostate, colon or rectal cancer. In still another aspect, the invention provides a method of treating colorectal cancer in a subject in need thereof by administering to the subject a pharmaceutical composition comprising Avastin and a low dose of nitroglycerin, wherein the nitroglycerin is administered transdermally, and wherein the cancer is breast, prostate, colon or rectal cancer.

In another aspect, provided herein is a combination therapy, comprising and an effective amount of an anti-VEGF antibody and a nitric oxide mimetic. The combination therapy can be for the treatment of cancer. In one embodiment, the anti-VEGF antibody is Avastin and the nitric oxide mimetic is nitroglycerin. In still another embodiment, the nitroglycerin is administered at a low dose.

### DETAILED DESCRIPTION OF THE INVENTION

A growing body of evidence indicates that angiogenesis is essential to the progression of cancer. Angiogenesis is the sprouting of new capillaries from preexisting blood vessels. Normally, angiogenesis in mammals is confined to the reproductive system, embryogenesis and development, and repair after injury. However, angiogenesis can also occur in pathological conditions such as cancer, retinal neovascularization, neovascularization in atherosclerotic plaques, hemangiomas, arthritis, and psoriasis. Without vascularization, tumors may remain for years as small (less than a few millimeters) asymptomatic lesions. Angiogenesis allows the cancer cells access to the circulatory system. The new blood vessels also provide a gateway for cancer cells to enter the circulation and metastasize to distant sites.

Several approaches for inhibition of angiogenesis have been proposed as useful therapies for restricting tumor growth. These include inhibition of angiogenesis by (1) inhibition of release of “angiogenic molecules” such as VEGF (Vascular endothelial growth factor) and FGF (fibroblast growth factor), (2) neutralization of angiogenic molecules, such as by use of anti-bFGF antibodies, and (3) inhibition of the endothelial cell response to angiogenic stimuli. This latter strategy has received particular attention, and Folkman et al., Cancer Biology, 3:89 96 (1992), have described several endothelial cell response inhibitors, including collagenase inhibitor, basement membrane turnover inhibitors, angiostatic steroids, fungal-derived angiogenesis inhibitors, platelet factor 4, thrombospondin, arthritis drugs such as D-penicillamine and gold thiomalate, vitamin D analogs, alpha-interferon, and the like that might be used to inhibit angiogenesis.

Monoclonal antibodies (MAbs) to human tumor-associated differentiation antigens offer promise for the “targeting” of various antitumor agents such as radioisotopes, chemotherapeutic drugs, and toxins. In addition, some monoclonal antibodies have the advantage of killing tumor cells via antibody-dependent cellular cytotoxicity (ADCC) or complement-dependent cytotoxicity (CDC) in the presence of human effector cells or serum [Hellstrom et al., Proc. Natl. Acad. Sci. USA 83:7059 7063 (1986)], and there are a few monoclonal antibodies that have a direct antitumor activity that does not depend on any host component [Drebin et al., Oncogene 2:387 394 (1988)].

There is still a need, however, for novel and more effective angiogenesis modulation therapies for use alone or in combination with one or more of the currently available therapies for treatment of growth and proliferative disorders involving angiogenesis.

Anti-angiogenesis therapy (e.g., Avastin) has been shown to reduce tumor mass initially. However, because of its effect on the tumor vasculature, this form of therapy on its own results in tumor hypoxia. The latter leads to the selection of variants that exhibit resistance to hypoxia due activation of hypoxia-inducible genes, many of which are also linked to malignant phenotypes. Thus, recurrent lesions following anti-angiogenesis therapy are often highly aggressive. Concomitant nitric oxide mimetic therapy can interfere with the acquisition of cellular adaptations to hypoxia associated with malignant properties.

Administration of a low dose of a nitric oxide mimetic is sufficient to increase, restore or maintain levels of nitric oxide mimetic activity of cells so that a malignant cell phenotype is inhibited or prevented (see, e.g., U.S. Pat. No. 6,946,484, which is incorporated herein by reference). This inhibition and prevention occurs even when the cells are in a hypoxic environment and/or when combined with inhibition of endogenous nitric oxide production. Administration of very low doses of nitric oxide mimetics, even under conditions of markedly reduced levels of oxygen (1% O₂), was able to prevent the generation of a malignant cell phenotype and inhibit a malignant cell phenotype of cells.

Accordingly, the present invention is directed to a composition comprising NO mimetic molecules, NO-donating molecules or agents that stimulate NOS and maintain physiological NO levels, and an anti-angiogenesis agent. In one embodiment, the NO mimetic molecules, NO-donating molecules or agents that stimulate NOS are administered to a subject in need thereof in a low dose.

Pyruvate kinase type M2 (PKM2) is critical to the survival and proliferation of tumor cells and is involved in the switch in the tumor cells' metabolism from mitochondrial respiration to glycolysis (Warburg effect). Under the hypoxic conditions that invariably develop in solid tumors, glucose uptake (required for glycolysis) is dependent on the expression of the hypoxia-inducible glucose transporters (e.g., glut-1). Thus, activation of NO signaling following administration of NO mimetics can inhibit the hypoxic up-regulation of glucose transporter proteins thereby interfering with the Warburg effect and consequently survival of hypoxic tumor cells. Thus, in another aspect, the present invention is directed to a composition comprising mimetic molecules, NO-donating molecules or agents that stimulate NOS and maintain physiological NO levels, and a PKM2 blocker. In one embodiment, the NO mimetic molecules, NO-donating molecules or agents that stimulate NOS are administered to a subject in need thereof in a low dose.

In still another aspect, the present invention is directed to a composition comprising NO mimetic molecules, NO-donating molecules or agents that stimulate NOS and maintain physiological NO levels, and an anti-VEGF antibody for the treatment of cancer, such as adjuvant/non-metastatic colon cancer, metastatic breast cancer, metastatic renal cell carcinoma, metastatic glioblastoma multiforme, metastatic ovarian cancer, metastatic hormone-refractory prostate cancer, and metastatic or unresectable locally advanced pancreatic cancer. In particular embodiments, the compositions of the invention are used for the treatment of lung, prostate and colon cancer. In one embodiment, the NO mimetic molecules, NO-donating molecules or agents that stimulate NOS are administered to a subject in need thereof in a low dose.

In certain embodiments, the present invention is directed to a composition comprising a low dose of nitroglycerin and Avastin for the use of treatment of cancer in a subject in need thereof. In a particular embodiment, the low dose of nitroglycerin is administered using a transdermal patch.

In particular, specific embodiments of the invention are described herein as exemplary embodiments and are not intended to be limiting.

### Definitions

These and other embodiments of the invention will be described with reference to following definitions that, for convenience, are collected here.

For purposes of the present invention, the term “low dose” is meant an amount of nitric oxide mimetic that is capable of increasing, restoring or maintaining a level of nitric oxide mimetic activity to cells which inhibits or prevents cancer and cancer-related disorders. As will be understood by those of skill in the art upon reading this disclosure, the nitric oxide mimetic increases, restores or maintains activity both in and around the cell (i.e., in the cellular microenvironment).

Methods for determining levels of nitric oxide of cells based upon nitrite, nitrate and S-nitrosothiol levels in cell culture, as well as plasma and serum, have been described. Serum or plasma nitrate levels in healthy normal volunteers have been reported to show a mean nitric oxide level of 33.4±8.9 μM with a range of 14 to 60 μM (Marzinzig et al. Nitric Oxide: Biology and Chemistry 1987 1(2): 177-189). These levels, however, are based on NO synthase end products that accumulate and thus are likely to represent an overestimate of normal physiologic nitric oxide levels. Reported measured levels also vary depending upon the method selected for measurement. Further, levels of nitrite and nitrate in the plasma or serum are not solely representative of a patient's NO production. Based upon experiments described in U.S. Pat. No. 6,946,484, normal physiologic levels of nitric oxide mimetic activity of cells may be lower, for example at least 5-fold, and preferably 10- to 10,000-fold lower, than those reported in the art, depending upon the cell.

Short term nitric oxide mimetic therapy is generally administered at levels that increase nitric oxide mimetic activity of cells above normal physiologic levels. For purposes of the present invention, however, wherein longer term therapy is generally desired, induction of tolerance against the NO mimetic and side effects become concerns. Thus, in the present invention, the amount of nitric oxide mimetic administered is preferably very low so as to delay and/or reduce development of tolerance to the administered NO mimetic and/or unwanted side effects. For example, it is known that administration of nitric oxide or compounds which deliver nitric oxide to human beings at doses conventionally employed to treat cardiovascular conditions (i.e., GTN at 0.2 mg/h or greater) by vasodilation can provoke powerful vasodilator responses as well as development of drug tolerance against GTN upon repeated administration. Such administration is often accompanied by a number of undesirable side effects including headache, flushing and hypotension. In contrast, preferred doses of nitric oxide mimetic administered in the present invention to inhibit and prevent a malignant cell phenotype are lower, preferably at least 3 to 10,000-fold lower, more preferably at least 100- to at least 10,000-fold lower than those typically used in other therapeutic applications such as vasodilation and thus do not induce tolerance to the NO mimetic as quickly nor undesirable side effects. For example, using the nitric oxide mimetics sodium nitroprusside (SNP) and glyceryl trinitrate (GTN), it has been demonstrated that amounts ranging between 10⁻¹² and 10⁻¹⁰ M in the cellular environment can be used to prevent and inhibit a malignant cell phenotype. Further, doses of SNP as low as 10⁻¹⁴ M would be effective in preventing and inhibiting a malignant cell phenotype in less hypoxic or hyponitroxic environments. In certain embodiments, the low dose of the compositions of the invention is 3 to 10,000 fold lower than a dose of the composition that produces vasodilation.

Table 1 provides additional examples of various lower preferred doses for nitric oxide mimetics useful in the present invention as well as the comparative higher doses used in vasodilation therapy.

TABLE 1
Typical Vasodilatory and Microdoses of Organonitrate

| | | | Preferred Dose |
|---|---|---|---|
| | Commercial | | According to the |
| Compound | Product | Vasodilatory Dose | Present Invention |
| Nitroglycerin | Nitrostat ® (Parke- | Dissolve one tablet | Dissolve one tablet |
| (sublingual tablets) | Davis) 0.3 mg, 0.4 mg | (0.3-0.6 mg) | containing from |
| | and 0.6 mg | sublingually or in | about 0.02 μg to |
| | | the buccal pouch at | about 0.1 mg |
| | | the first sign of an | sublingually or in |
| | | acute anginal attack | the buccal pouch |
| Nitroglycerin | Nitrolingual ® Spray | One or two metered | About 0.02 μg to |
| (lingual aerosol) | (Rhone-Poulenc | (0.4-0.8 mg) | about 0.1 mg |
| | Rorer); metered | sprayed onto or | sprayed onto or |
| | aerosol, 0.4 mg/ | under the tongue at | under the tongue |
| | metered dose | the onset of an | |
| | | anginal attack | |
| Nitroglycerin | Minitran ® (3M | Suggested dose is | About 0.0125 μg/hr-0.1 mg/h |
| (transdermal patch) | Corporation); | between 0.2-0.8 mg/h | |
| | Transdermal patches | for 12-14 h | |
| | having the following | daily with a | |
| | characteristics (size | minimum nitrate- | |
| | (cm2), delivery rate | free interval of 10-12 h | |
| | (mg/h)); (3.3, 0.1; | | |
| | 6.7, 0.2; 13.3, 0.4; | | |
| | and 20.0, 0.6) | | |
| Nitroglycerin | NITRO-BID ® | Doses used in | Ointment |
| (ointment) | Ointment (Hoechst | clinical trials have | containing about |
| | Marion Roussel); | ranged from ½ inch | 3.75 mg of |
| | lactose and 2% | (1.3 cm; 7.5 mg), to | nitroglycerin |
| | nitroglycerin in a | 2 inches (5.1 cm; 30 mg), | applied to the arms |
| | base of lanolin and | typically | or legs over an area |
| | white petrolatum. | applied to 36 square | of about 36 square |
| | Each inch (2.5 cm), | inches (232 square | inches (232 cm²) |
| | as squeezed from | cm) of skin on the | |
| | the tube, contains | arms or legs | |
| | approximately 15 mg | | |
| | of nitroglycerin | | |
| Isosorbide 5- | IMSO ® (Wyeth- | 20 mg twice daily | About 1 μg to about |
| mononitrate | Ayerst) 20 mg | Chronic (Adults): | 2.5 mg twice daily |
| Erythrityl | tablets Cardilate ® | 10 mg orally 4 | Chronic (Adults): |
| tetranitrate | (Burroughs- | times daily, | About 0.5 μg to |
| | Wellcome); | gradually increased | about 1.25 mg |
| | oral/sublingual | to 20 mg, if | orally 4 times daily, |
| | tablets, 5 mg, 10 mg | necessary, not to | gradually increased |
| | | exceed 100 mg/day. | to about 1 μg to |
| | | | about 2.5 mg/day, if |
| | | | necessary, not to |
| | | | exceed about 5 to |
| | | | about 12.5 mg/day |
| Sodium | Nipride ® (Roche); | Slow infusion at a | Slow infusion at a |
| nitroprusside | Nitropress ® | rate of 0.5 μg/kg/min | rate of from 0.025 ng/kg/min |
| | (Abbott); | of a | to about |
| | intravenous solution | solution of 50 mg in | 0.063 μg/kg/min of |
| | | 500-1000 mL of 5% | a solution of 50 mg |
| | | dextrose up to a | in 500-1000 mL of |
| | | limit of 3.5 mg/kg | 5% dextrose up to a |
| | | in brief infusions | limit of about 0.18 mg/kg |
| | | | to about 0.44 mg/kg |
| | | | in brief |
| | | | infusions |
| Molsidomine | Corvaton ® (Hoechst | 2 mg/day up to 36 mg/ | 0.1 μg/day up to 4.5 mg/ |
| | Marion Roussel); 2 mg, | day given in | day given in |
| | 4 mg, and 6 mg | separate doses | separate doses |
| | tablets | either twice or three | either twice or three |
| | | times daily | times daily |
| Nicorandil | Nicorandil ® | For the treatment of | About 0.5 μg to |
| | (Chugai | angina 10-20 mg | about 1 mg twice |
| | Pharmaceuticals, | twice daily | daily |
| | Japan), Dancor ® | | |
| | (Merck) 10 mg, 20 mg | | |
| | tablets | | |

As used herein, the term “anti-angiogenesis drug” or “anti-angiogenesis agent” includes any compound or therapy that can impede or stop, either permanently or temporarily, the progression of angiogenesis in a subject. To determine the ability of a compound to inhibit angiogenesis, conventional in vitro and in vivo assays can be used. “Anti-angiogenesis drug” or “anti-angiogenesis agent” includes, but is not limited to, bevacizumab (Avastin), lucentis (ranibizumab, a humanized anti-VEGF antibody fragment that inhibits activity), macugen (pegaptanib sodium injection), angiostatin, endostatin, platelet factor-4, platelet factor-4-derived molecules, vascular endothelial growth inhibitor (VEGI), trastuzumab (Herceptin), sunitinib (SU011248), lenalidomide, thalidomide, pazopanib, sorafenib (Nexavar), or axitinib. In a particular embodiment, the anti-angiogenesis agent includes bevacizumab (avastin), ranibizumab (lucentis), sunitinib (sutent), sorafenib (nexavar), axitinib or pazopanib.

Bevacizumab is a monoclonal antibody that has shown significant activity in colon, breast, lung and ovarian cancer. It is sold under the tradename Avastin (Genentech), and described in U.S. Pat. No. 6,054,297 (incorporated herein by reference in its entirety). The antibody targets vascular endothelial growth factor (VEGF), a protein made by cells that stimulates the production of new blood vessels. VEGF is structurally related to platelet-derived growth factor (PDGF). The gene is located on chromosome 6 p12.

As used herein, the term “pyruvate kinase type M2 blocker” or “PKM2 blocker” refers to a compound that inhibits or otherwise alters, the activity of the isoenzyme PKM2 (also known as M2-PK). To determine the ability of a compound to inhibit PKM2, conventional in vitro and in vivo assays can be used.

The term “cancer” is interpreted broadly. The composition of the present invention (i.e., a composition of a low dose of a NO mimetic molecule and an anti-VEGF antibody, e.g., a low dose of nitroglycerin and Avastin) can be an “anti-cancer agent,” which term also encompasses “anti-tumor cell growth agent” and “anti-neoplastic agent.” For example, the methods of the invention are useful for treating cancers and radiosensitizing tumor cells in cancers such as ACTH-producing tumors, acute lymphocytic leukemia, acute nonlymphocytic leukemia, cancer of the adrenal cortex, bladder cancer, brain cancer, breast cancer, cervical cancer, chronic lymphocytic leukemia, chronic myelocytic leukemia, colorectal cancer, cutaneous T-cell lymphoma, endometrial cancer, esophageal cancer, Ewing's sarcoma, gallbladder cancer, hairy cell leukemia, head and neck cancer, Hodgkin's lymphoma, Kaposi's sarcoma, kidney cancer, liver cancer, lung cancer (small and/or non-small cell), malignant peritoneal effusion, malignant pleural effusion, melanoma, mesothelioma, multiple myeloma, neuroblastoma, non-Hodgkin's lymphoma, osteosarcoma, ovarian cancer, ovary (germ cell) cancer, prostate cancer, pancreatic cancer, penile cancer, retinoblastoma, skin cancer, soft-tissue sarcoma, squamous cell carcinomas, stomach cancer, testicular cancer, thyroid cancer, trophoblastic neoplasms, uterine cancer, vaginal cancer, cancer of the vulva and Wilm's tumor. In particular embodiments, the compositions of the invention are used for the treatment of lung, prostate and colon cancer. In other embodiments, the cancer to be treated is breast cancer, esophageal cancer, gastrointestinal stromal tumors (GIST), kidney (renal cell) cancer, leukemia, liver (adult primary) cancer, lymphoma, melanoma, multiple myeloma, non-small cell lung cancer (NSCLC), ovarian epithelial cancer, pancreatic cancer, prostate cancer, or stomach (gastric) cancer.

In another embodiment, the compositions of the invention are effective in the treatment of micrometastasis. Micrometastasis is a form of metastasis (the spread of a cancer from its primary location to a distant site) in which secondary tumors are too minuscule to be detected.

The term “treatment” or “treating,” as used herein, is defined as the application or administration of a therapeutic agent, i.e., a composition of the invention (e.g., a composition comprising a low dose of nitroglycerin and Avastin), to a subject, or application or administration of a therapeutic agent to an isolated tissue or cell line from a subject (e.g., for diagnosis or ex vivo applications), who suffers from cancer, cancer-related disorders, and any other disease or disorder described herein, with the purpose to cure, heal, alleviate, relieve, alter, remedy, ameliorate, improve or affect the disease or disorder, the symptoms of the disease or disorder or the disease or disorder itself. Such treatments may be specifically tailored or modified, based on knowledge obtained from the field of pharmacogenomics.

The term “subject” includes living organisms in which cancer and cancer-related disorders can occur, or which are susceptible to these disorders or any other disorder disclosed herein. The term “subject” includes animals (e.g., mammals, e.g., cats, dogs, horses, pigs, cows, goats, sheep, rodents, e.g., mice or rats, rabbits, squirrels, bears, primates (e.g., chimpanzees, monkeys, gorillas, and humans)), as well as chickens, ducks, geese, and transgenic species thereof; and cells, e.g., immortalized or nonimmortalized cells, derived therefrom. In a particular embodiment, the subject is a human.

Administration of the compositions of the present invention to a subject to be treated can be carried out using known procedures, at dosages and for periods of time effective to treat the diseases and disorders of the invention. An effective amount of the composition necessary to achieve a therapeutic effect may vary according to factors such as the state of the disease or disorder in the subject, the age, sex, and weight of the subject, and the ability of the therapeutic compound to inhibit the disease or disorder in the subject. Dosage regimens can be adjusted to provide the optimum therapeutic response. For example, several divided doses may be administered daily or the dose may be proportionally reduced as indicated by the exigencies of the therapeutic situation. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

In particular, the selected dosage level will depend upon a variety of factors including the activity of the particular compound of the present invention employed, the time of administration, the rate of excretion of the particular compound being employed, the duration of the treatment, other drugs, compounds or materials used in combination with the particular compound employed, the age, sex, weight, condition, general health and prior medical history of the patient being treated, and like factors well known in the medical arts.

A medical doctor, e.g., physician or veterinarian, having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

The regimen of administration can affect what constitutes an effective amount. The therapeutic formulations can be administered to the subject either prior to or after the onset of a disease or disorder disclosed herein. Further, several divided dosages, as well as staggered dosages, can be administered daily or sequentially, or the dose can be continuously infused, or can be a bolus injection. Further, the dosages of the therapeutic formulations can be proportionally increased or decreased as indicated by the exigencies of the therapeutic or prophylactic situation.

In particular embodiments, it is especially advantageous to formulate compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The specification for the dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of, for example, cancer in subjects.

### Compounds of the Invention

The present invention is directed to a composition comprising low doses of NO mimetic molecules, low doses of NO-donating molecules or low doses of agents that stimulate NOS and maintain physiological NO levels, and an anti-VEGF antibody.

The low doses of NO mimetic molecules, low doses of NO-donating molecules or low doses of NOS-stimulating agents of the invention, can be prepared using techniques well-known to one of skill in the art. Similar compositions are described in U.S. Pat. Nos. 6,946,484, 6,165,975 and 6,423,683, all of which are incorporated herein by reference in their entirety.

For purposes of the present invention, by the terms “NO mimetic,” “NO mimetic molecule” or “NO-donating molecule,” it is meant nitric oxide, or a functional equivalent thereof; any compound which mimics the effects of nitric oxide, generates or releases nitric oxide through biotransformation, generates nitric oxide spontaneously, or spontaneously releases nitric oxide; any compound which in any other manner generates nitric oxide or a nitric oxide-like moiety or activates other stages of the NO pathway; or any compound which enables or facilitates NO utilization by the cell, when administered to an animal. Such compounds can also be referred to as “NO donors,” “NO prodrugs,” “NO producing agents,” “NO delivering compounds,” “NO generating agents,” “NO mimetic molecules,” “NO donating molecule,” an agent that stimulates NOS and maintains physiological NO and “NO providers.” Examples of such compounds include, but are not limited to: organonitrates such as nitroglycerin (GTN), isosorbide 5-mononitrate (ISMN), isosorbide dinitrate (ISDN), pentaerythritol tetranitrate (PETN), erthrityl tetranitrate (ETN); amino acid derivatives such as N-hydroxyl-L-arginine (NOHA), N⁶-(1-iminoethyl)lysine) (L-NIL), L-N⁶-(1-iminoethyl)ornithine (LN-NIO), N¹⁰⁶-methyl-L-arginine (L-NMMA), and S-nitrosoglutathione (SNOG); and other compounds which generate or release NO under physiologic conditions such as S,S-dinitrosodithiol (SSDD), [N-[2-(nitroxyethyl)]-3-pyridinecarboxamide (nicorandil), sodium nitroprus side (SNP), S-nitroso-N-acetylpenicilamine (SNAP), 3-morpholino-sydnonimine (SIN-1), molsidomine, DEA-NONOate(2-(N,N-diethylamino)-diazenolate-2-oxide), and spermine NONOate (N-[4-[1-(3-aminopropyl)-2-hydroxy-2-nitrosohydrazino]butyl-1,3-propanediamine). Organic nitrates GTN, ISMN, ISDN, ETN, and PETN, as well as nicorandil (commonly known as a potassium channel opener) are commercially available in pharmaceutical dosage forms. SIN-1, SNAP, S-thioglutathione, L-NMMA, L-NIL, L-NIO, spermine NONOate, and DEA-NONOate are commercially available from Biotium, Inc. Richmond, Calif. As used herein the term “nitric oxide mimetic” is also intended to mean any compound which acts as a nitric oxide pathway mimetic, that has nitric oxide-like activity, or that mimics the effect of nitric oxide. Such compounds may not necessarily release, generate or provide nitric oxide, but they have a similar effect to nitric oxide on a pathway that is affected by nitric oxide. For example, nitric oxide has both cyclic GMP-dependent and cyclic GMP-independent effects. Nitric oxide is known to activate the soluble form of guanylyl cyclase thereby increasing intracellular levels of the second messenger cyclic GMP and other interactions with other intracellular second messengers such as cyclic AMP. As such, compounds which directly activate either particulate or soluble guanylyl cyclase such as natriuretic peptides (ANP, BNP, and CNP), 3-(5′-hydroxymethyl-2′ furyl)-1-benzyl indazole (YC-cGMP or YC-1) and 8-(4-chlorophenylthio)guanosine 3′,5′-cyclic monophosphate (8-PCPT-cGMP), are also examples of NO-mimetics. In some embodiments of the present invention, however, it is preferred that the NO-mimetic not encompass a compound which directly activates either particulate or soluble guanylyl cyclase. Nitric oxide mimetic activity encompasses those signal transduction processes or pathways which comprise at least one NO mimetic-binding effector molecule, such as for example, guanylyl cyclase and other heme containing proteins. Example of agents which function as NO mimetics by enabling or facilitating NO utilization by the cell are compounds which inhibit phosphodiesterase activity and/or expression, such as phosphodiesterase inhibitors.

In a certain embodiment of the present invention, more than one NO mimetic is administered in the composition of the invention. In this embodiment, it is preferred that the NO mimetics target or act upon different parts of the NO pathway of the cell. For example, an NO donor can be co-administered with a compound that inhibits cyclic nucleotide (e.g., cAMP or cGMP) degradation such as a phosphodiesterase inhibitor. Preferred phosphodiesterase (PDE) inhibitors useful as NO mimetics are those inhibiting PDE-1 through PDE-5.

In a preferred embodiment, the NO mimetic molecule is GTN, which is used for the treatment of cancer in a human. In a particular embodiment, a low dose of GTN is used to treat prostate, breast, colon or rectal cancer in a human.

Although Avastin (Bevacizumab) is perhaps the most common anti-VEGF antibody for cancer treatment, the instant invention is directed toward any anti-VEGF antibody with the same or similar activity to Bevacizumab. Antibodies with the same or similar activity can be described as “biologically equivalent.” Methods to make and identify such antibodies are described in the art.

In one aspect, the anti-VEGF antibody binds to any region or epitope on VEGF. In an alternative embodiment, the antibody binds to the same epitope on VEGF as

Avastin. In a further aspect, the antibody is a variant or derivative of Avastin of any of the above and can include function fragments, derivatives or such antibodies conjugated to other agents.

### Combination Therapies

The compositions of the present invention are intended to be useful, e.g., in the methods of present invention, in combination with one or more additional compounds useful for treating cancer. These additional compounds may comprise compounds of the present invention or compounds, e.g., commercially available compounds, known to treat, prevent, or reduce the symptoms of cancer.

In particular, the compositions of the invention, e.g., low dose nitroglycerin and Avastin, can be co-administered with compounds used for the treatment of cancer, including, but not limited to, taxol, 5-fluorouricil, paclitaxel, bolus-IFL, FOLFOX4 and carboplatin.

A combination of compounds described herein can either result in synergistic increase in effectiveness against, for example, cancer, relative to effectiveness following administration of each compound when used alone, or such an increase can be additive. Compositions described herein typically include lower dosages of each compound in a composition, thereby avoiding adverse interactions between compounds and/or harmful side effects, such as ones which have been reported for similar compounds. Furthermore, normal amounts of each compound when given in combination could provide for greater efficacy in subjects who are either unresponsive or minimally responsive to each compound when used alone.

A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

### Formulations for Administration

In another embodiment, the present invention is directed to a packaged pharmaceutical composition comprising a container holding a therapeutically effective amount of a composition of the invention; and compositions for using the compound to treat, prevent, or reduce one or more symptoms of cancer.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions can contain information pertaining to the compound's ability to perform its intended function, e.g., treating, preventing, or reducing one or more lipoprotein abnormalities in a subject.

Another embodiment of the invention is a pharmaceutical composition comprising a therapeutically effective amount of a composition of the invention, e.g., GTN and Avastin, and a pharmaceutically acceptable carrier.

The language “therapeutically effective amount” describes the low dose amount of composition of the invention that is effective to treat cancer.

The term “pharmaceutical composition” refers to a composition comprising an entity to be delivered, wherein the carrier is a pharmaceutically acceptable carrier.

The language “pharmaceutically acceptable carrier” includes a pharmaceutically acceptable material, composition or carrier, such as a liquid or solid filler, diluent, excipient, solvent or encapsulating material, involved in carrying or transporting a compound(s) of the present invention within or to the subject such that it can perform its intended function. Typically, such compounds are carried or transported from one organ, or portion of the body, to another organ, or portion of the body. Each carrier must be “acceptable” in the sense of being compatible with the other ingredients of the formulation, and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. As used herein “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds can also be incorporated into the compositions.

The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin. In one embodiment, the pharmaceutically acceptable carrier is not DMSO alone.

The compounds for use in the invention can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For example, for oral administration the compounds can be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets can be coated using suitable methods and coating materials such as OPADRY™ OY film coating systems available from Colorcon, West Point, Pa. (e.g., OPADRY™ OY Type, OY-C Type, Organic Enteric OY-P Type, Aqueous Enteric OY-A Type, OY-PM Type and OPADRY™ White, 32K18400). Liquid preparation for oral administration can be in the form of solutions, syrups or suspensions. The liquid preparations can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds for use in the method of the invention can be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents can be used.

### Transmucosal Administration

Transmucosal administration is carried out using any type of formulation or dosage unit suitable for application to mucosal tissue. For example, the selected active agent can be administered to the buccal mucosa in an adhesive tablet or patch, sublingually administered by placing a solid dosage form under the tongue, lingually administered by placing a solid dosage form on the tongue, administered nasally as droplets or a nasal spray, administered by inhalation of an aerosol formulation, a non-aerosol liquid formulation, or a dry powder, placed within or near the rectum (“transrectal” formulations), or administered to the urethra as a suppository, ointment, or the like.

### Transurethal Administration

With regard to transurethal administration, the formulation can comprise a urethral dosage form containing the active agent and one or more selected carriers or excipients, such as water, silicone, waxes, petroleum jelly, polyethylene glycol (“PEG”), propylene glycol (“PG”), liposomes, sugars such as mannitol and lactose, and/or a variety of other materials. A transurethral permeation enhancer can be included in the dosage from. Examples of suitable permeation enhancers include dimethylsulfoxide (“DMSO”), dimethyl formamide (“DMF”), N,N-dimethylacetamide (“DMA”), decylmethylsulfoxide (“C10 MSO”), polyethylene glycol monolaurate (“PEGML”), glycerol monolaurate, lecithin, the 1-substituted azacycloheptan-2-ones, particularly 1-n-dodecylcyclazacycloheptan-2-one (available under the trademark Azone™ from Nelson Research & Development Co., Irvine, Calif.), SEPA™ (available from Macrochem Co., Lexington, Mass.), surfactants as discussed above, including, for example, Tergitol™, Nonoxynol-9™ and TWEEN-80™, and lower alkanols such as ethanol.

### Transrectal Administration

Transrectal dosage forms may include rectal suppositories, creams, ointments, and liquid formulations (enemas). The suppository, cream, ointment or liquid formulation for transrectal delivery comprises a therapeutically effective amount of the selected active agent and one or more conventional nontoxic carriers suitable for transrectal drug administration. The transrectal dosage forms of the present invention can be manufactured using conventional processes. The transrectal dosage unit can be fabricated to disintegrate rapidly or over a period of several hours. The time period for complete disintegration may be in the range of from about 10 minutes to about 6 hours, e.g., less than about 3 hours.

### Vaginal or Perivaginal Administration

Vaginal or perivaginal dosage forms may include vaginal suppositories, creams, ointments, liquid formulations, pessaries, tampons, gels, pastes, foams or sprays. The suppository, cream, ointment, liquid formulation, pessary, tampon, gel, paste, foam or spray for vaginal or perivaginal delivery comprises a therapeutically effective amount of the selected active agent and one or more conventional nontoxic carriers suitable for vaginal or perivaginal drug administration. The vaginal or perivaginal forms of the present invention can be manufactured using conventional processes as disclosed in Remington: The Science and Practice of Pharmacy, supra (see also drug formulations as adapted in U.S. Pat. Nos. 6,515,198; 6,500,822; 6,417,186; 6,416,779; 6,376,500; 6,355,641; 6,258,819; 6,172,062; and 6,086,909). The vaginal or perivaginal dosage unit can be fabricated to disintegrate rapidly or over a period of several hours. The time period for complete disintegration may be in the range of from about 10 minutes to about 6 hours, e.g., less than about 3 hours.

### Intranasal or Inhalation Administration

The active agents may also be administered intranasally or by inhalation. Compositions for intranasal administration are generally liquid formulations for administration as a spray or in the form of drops, although powder formulations for intranasal administration, e.g., insufflations, nasal gels, creams, pastes or ointments or other suitable formulators can be used. For liquid formulations, the active agent can be formulated into a solution, e.g., water or isotonic saline, buffered or unbuffered, or as a suspension. In certain embodiments, such solutions or suspensions are isotonic relative to nasal secretions and of about the same pH, ranging e.g., from about pH 4.0 to about pH 7.4 or, from about pH 6.0 to about pH 7.0. Buffers should be physiologically compatible and include, for example, phosphate buffers. Furthermore, various devices are available in the art for the generation of drops, droplets and sprays, including droppers, squeeze bottles, and manually and electrically powered intranasal pump dispensers. Active agent containing intranasal carriers can also include nasal gels, creams, pastes or ointments with a viscosity of, e.g., from about 10 to about 6500 cps, or greater, depending on the desired sustained contact with the nasal mucosal surfaces. Such carrier viscous formulations may be based upon, for example, alkylcelluloses and/or other biocompatible carriers of high viscosity well known to the art (see e.g., Remington: The Science and Practice of Pharmacy, supra). Other ingredients, such as preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odor for the formulation. Formulations for inhalation may be prepared as an aerosol, either a solution aerosol in which the active agent is solubilized in a carrier (e.g., propellant) or a dispersion aerosol in which the active agent is suspended or dispersed throughout a carrier and an optional solvent. Non-aerosol formulations for inhalation can take the form of a liquid, typically an aqueous suspension, although aqueous solutions may be used as well. In such a case, the carrier is typically a sodium chloride solution having a concentration such that the formulation is isotonic relative to normal body fluid. In addition to the carrier, the liquid formulations can contain water and/or excipients including an antimicrobial preservative (e.g., benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate and combinations thereof), and/or a suspending agent (e.g., agar, bentonite, microcrystalline cellulose, sodium carboxymethylcellulose, hydroxypropyl methylcellulose, tragacanth, veegum and combinations thereof). Non-aerosol formulations for inhalation can also comprise dry powder formulations, particularly insufflations in which the powder has an average particle size of from about 0.1 μm to about 50 μm, e.g., from about 1 μm to about 25 μm.

### Topical Formulations

Topical formulations can be in any form suitable for application to the body surface, and may comprise, for example, an ointment, cream, gel, lotion, solution, paste or the like, and/or may be prepared so as to contain liposomes, micelles, and/or microspheres. In certain embodiments, topical formulations herein are ointments, creams and gels.

### Transdermal Administration

The composition of the invention may also be administered through the skin or mucosal tissue using conventional transdermal drug delivery systems, wherein the agent is contained within a laminated structure (typically referred to as a transdermal “patch”) that serves as a drug delivery device to be affixed to the skin. Transdermal drug delivery may involve passive diffusion or it may be facilitated using electrotransport, e.g., iontophoresis. In a typical transdermal patch, the drug composition is contained in a layer, or “reservoir,” underlying an upper backing layer. The laminated structure may contain a single reservoir, or it may contain multiple reservoirs. In one type of patch, referred to as a “monolithic” system, the reservoir is comprised of a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are separate and distinct layers, with the adhesive underlying the reservoir which, in this case, may be either a polymeric matrix as described above, or it may be a liquid or hydrogel reservoir, or may take some other form.

### Intrathecal Administration

One common system utilized for intrathecal administration is the APT Intrathecal treatment system available from Medtronic, Inc. APT Intrathecal uses a small pump that is surgically placed under the skin of the abdomen to deliver medication directly into the intrathecal space. The medication is delivered through a small tube called a catheter that is also surgically placed. The medication can then be administered directly to cells in the spinal cord involved in conveying sensory and motor signals associated with lower urinary tract disorders.

### Intravesical Administration

The term intravesical administration is used herein in its conventional sense to mean delivery of a drug directly into the bladder. Suitable methods for intravesical administration can be found, for example, in U.S. Pat. Nos. 6,207,180 and 6,039,967.

### Additional Administration Forms

Additional dosage forms of this invention include dosage forms as described in U.S. Pat. No. 6,340,475, U.S. Pat. No. 6,488,962, U.S. Pat. No. 6,451,808, U.S. Pat. No. 5,972,389, U.S. Pat. No. 5,582,837, and U.S. Pat. No. 5,007,790. Additional dosage forms of this invention also include dosage forms as described in U.S. patent application Ser. No. 20030147952, U.S. patent application Ser. No. 20030104062, U.S. patent application Ser. No. 20030104053, U.S. patent application Ser. No. 20030044466, U.S. patent Application Ser. No. 20030039688, and U.S. patent application Ser. No. 20020051820. Additional dosage forms of this invention also include dosage forms as described in PCT Patent Application WO 03/35041, PCT Patent Application WO 03/35040, PCT Patent Application WO 03/35029, PCT Patent Application WO 03/35177, PCT Patent Application WO 03/35039, PCT Patent Application WO 02/96404, PCT Patent Application WO 02/32416, PCT Patent Application WO 01/97783, PCT Patent Application WO 01/56544, PCT Patent Application WO 01/32217, PCT Patent Application WO 98/55107, PCT Patent Application WO 98/11879, PCT Patent Application WO 97/47285, PCT Patent Application WO 93/18755, and PCT Patent Application WO 90/11757.

### Controlled Release Formulations and Drug Delivery Systems

In certain embodiments, the formulations of the present invention can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time can be as long as a month or more and should be a release which is longer that the same amount of agent administered in bolus form.

For sustained release, the compounds can be formulated with a suitable polymer or hydrophobic material which provides sustained release properties to the compounds. As such, the compounds for use the method of the invention can be administered in the form of microparticles for example, by injection or in the form of wafers or discs by implantation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes after drug administration.

In particular embodiments, the low dose nitric oxide mimetic (e.g., nitroglycerin) is administered to the subject in need thereof using a transdermal patch, while the anti-VEGF antibody (e.g., Avastin) is administered to the subject in need thereof intravenously.

Additional methods of administration of Avastin are described in U.S. Pat. No. 6,054,297, which is incorporated herein by reference. Additional methods of administration of a low dose of a nitric oxide mimetic are described in U.S. Pat. Nos. 6,946,484, 6,165,975 and 6,423,683, all of which are incorporated herein by reference in their entirety.

### Dosing

The therapeutically effective amount or dose of a compound of the present invention will depend on the age, sex and weight of the patient, the current medical condition of the patient and the nature of the cancer being treated. The skilled artisan will be able to determine appropriate dosages depending on these and other factors.

A suitable dose of a composition of the present invention is lower, preferably at least 3 to 10,000-fold lower, more preferably at least 100- to at least 10,000-fold lower than what is typically used in other therapeutic applications such as vasodilation. This includes a ranges between 10⁻¹² and 10⁻¹⁰ M, 10⁻¹⁴ to 10⁻¹⁰, and 10⁻¹⁴ to 10⁻¹⁰ in the cellular environment can be used to prevent and inhibit a malignant cell phenotype. In certain embodiments, the low dose of the compositions of the invention is 3 to 10,000 fold lower than a dose of the composition that produces vasodilation. Stated alternatively, the low dose of the compositions of the invention will be 0.0001% to 33%, e.g., 0.001% to 33%, of the commercial dose of a NO mimetic product that is used to induce vasodilation. For example, as stated in Table 1, Nitrolingual® spray consists of 0.4 mg/metered dose for the purposes of inducing vasodilation. For purposes of the instant invention, the same nitroglycerin spray would be administered in a “low dose” of 0.02 μg to 0.1 mg (i.e., 0.005%-25% of the Nitrolingual® commercial dose) for the purposes of treating cancer.

A suitable low dose of the compositions of the invention, e.g., GTN, can be, for example, in the range of 0.01 ng-20 mg, e.g., 0.20 ng-10 mg, e.g., 0.025 ng-4.5 mg, e.g., 0.0125 μg-3.75 mg, e.g., 0.02 μg-2.5 mg, e.g., 0.063 μg-1 mg, and, e.g., 0.1 μg-0.5 μg.

Suitable doses of anti-VEGF antibodies are known in the art. For example, the recommended dose of Avastin is 1-30 mg/kg, e.g., 15 mg/kg, for example, as an IV infusion every 3 weeks. Avastin, used in combination with intravenous 5-FU-based chemotherapy, is administered as an intravenous infusion (1-20 mg/kg, e.g., 5 mg/kg or 10 mg/kg), for example, every 14 days. The recommended dose of Avastin, when used in combination with bolus-IFL, is 1-20 mg/kg, e.g., 5 mg/kg. The recommended dose of Avastin, when used in combination with FOLFOX4, is 1-20 mg/kg, e.g., 10 mg/kg.

In one embodiment, the pharmaceutical composition provided herein can comprise the transdermal delivery of low dose GTN, and the intravenous administration of Avastin to a subject. The administration to the subject can occur at substantially the same time, or within 24 hours of one another.

It is understood that the amount of composition dosed per day can be administered every day, every other day, every 2 days, every 3 days, every 4 days, every 5 days, etc. For example, with every other day administration, a 5 mg per day dose can be initiated on Monday with a first subsequent 5 mg per day dose administered on Wednesday, a second subsequent 5 mg per day dose administered on Friday, etc.

The compounds for use in the method of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

### Exemplification

Methods of preparing and testing Avastin for the treatment of cancer are disclosed in U.S. Pat. No. 6,054,297, which is incorporated herein by reference in its entirety. A clinical trial demonstrating the effectiveness of Avastin against colorectal cancer can be found here: http://www.cancer.gov/clinicaltrials/results/bevacizumab-and-colorectal-cancer0601. A clinical trial demonstrating the effectiveness of Avastin against breast cancer can be found here:

http://www.nci.nih.gov/newscenter/pressreleases/AvastinBreast. A clinical trial demonstrating the effectiveness of Avastin against lung cancer can be found here: http://www.nci.nih.gov/newscenter/pressreleases/AvastinLung. All of these websites are incorporated herein by reference in their entirety.

Methods of preparing and testing transdermal delivery of low dose nitroglycerin for the treatment of cancer are disclosed in U.S. Pat. No. 6,946,484, which is incorporated herein by reference in its entirety.

### Equivalents

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including reaction times, reaction size/volume, and experimental reagents, such as solvents, catalysts, pressures, atmospheric conditions, e.g., nitrogen atmosphere, and reducing/oxidizing agents, etc., with art-recognized alternatives and using no more than routine experimentation, are within the scope of the present application.

It is to be understood that wherever values and ranges are provided herein, e.g., in ages of subject populations, dosages, and blood levels, all values and ranges encompassed by these values and ranges, are meant to be encompassed within the scope of the present invention. Moreover, all values that fall within these ranges, as well as the upper or lower limits of a range of values, are also contemplated by the present application.

### Incorporation by Reference

The contents of all references, issued patents, and published patent applications cited throughout this application are hereby expressly incorporated by reference in their entireties. It should be understood that the use of any of the compounds described herein are within the scope of the present invention and are intended to be encompassed by the present invention and are expressly incorporated herein for all purposes.

1-3. (canceled)

4. A pharmaceutical composition comprising an anti-angiogenesis agent and a nitric oxide mimetic.

5. The pharmaceutical composition of claim 4, wherein the anti-angiogenesis agent includes bevacizumab (avastin), lucentis (ranibizumab), macugen (pegaptanib sodium injection), angiostatin, endostatin, platelet factor-4, platelet factor-4-derived molecules, vascular endothelial growth inhibitor (VEGI), trastuzumab (Herceptin), sunitinib (SU011248), lenalidomide, thalidomide, pazopanib, sorafenib (Nexavar), or axitinib.

6. The pharmaceutical composition of claim 4, wherein the anti-angiogenesis agent includes bevacizumab (avastin), ranibizumab (lucentis), sunitinib (sutent), sorafenib (nexavar), axitinib or pazopanib.

7. A pharmaceutical composition comprising a pyruvate kinase type M2 (PKM2) blocker and a nitric oxide mimetic.

8. The pharmaceutical composition of claims 4 or 7, wherein the nitric oxide mimetic is selected from the group consisting of nitroglycerin (GTN), isosorbide 5-mononitrate (ISMN), isosorbide dinitrate (ISDN), pentaerythritol tetranitrate (PETN), erthrityl tetranitrate (ETN), N-hydroxyl-L-arginine (NOHA), N⁶-(1-iminoethyl)lysine) (L-NIL), L-N⁶-(1-iminoethyl)ornithine (LN-NIO), N⁶-methyl-L-arginine (L-NMMA), and S-nitrosoglutathione (SNOG).

9. The pharmaceutical composition of claims 4 or 7, wherein the nitric oxide mimetic is selected from the group consisting of S,S-dinitrosodithiol (SSDD), [N-[2-(nitroxyethyl)]-3-pyridinecarboxamide (nicorandil), sodium nitroprusside (SNP), S-nitroso-N-acetylpenicilamine (SNAP), 3-morpholino-sydnonimine (SIN-1), molsidomine, DEA-NONOate(2-(N,N-diethylamino)-diazenolate-2-oxide), and spermine NONOate (N-[4-[1-(3-aminopropyl)-2-hydroxy-2-nitrosohydrazino]butyl-1,3-propanediamine).

10. The pharmaceutical composition of claim 9, wherein the nitric oxide mimetic is nitroglycerin (GTN).

11. (canceled)

12. The pharmaceutical composition of claim 9, wherein the nitric oxide mimetic is administered in a low dose.

13-15. (canceled)

16. A method of treating cancer in a subject in need thereof by administering to the subject a pharmaceutical composition comprising an anti-angiogenesis agent and a nitric oxide mimetic.

17. A method of treating cancer in a subject in need thereof by administering to the subject a pharmaceutical composition comprising a pyruvate kinase type M2 (PKM2) blocker and a nitric oxide mimetic.

18. The method of claims 16 or 17, wherein the cancer is selected from the group consisting of breast, ovarian, prostate, lung, colon, and rectal cancer, or metastatic carcinoma of the colon or rectum.

19. The method of claims 16 or 17, wherein the cancer is recurrent or metastatic non-squamous, non-small cell lung cancer.

20-24. (canceled)

25. The method of claims 16 or 17, wherein the nitric oxide mimetic is administered in a low dose.

26. (canceled)

27. (canceled)

28. A method of treating cancer in a subject in need thereof by administering to the subject a pharmaceutical composition comprising Avastin and a low dose of nitroglycerin, wherein the nitroglycerin is administered transdermally.

29-33. (canceled)
